(19) United States
(12) Patent Application Publication
(10) Pub. No.: US 2022/0001074 A1
(43) Pub. Date: Jan. 6, 2022

(54) Bioadhesive for Soft Tissue Repair

(71) Applicants: The Schepens Eye Research Institute, Inc., Boston, MA (US); Northeastern University, Boston, MA (US)

(72) Inventors: Reza Dana, Newton, MA (US); Ahmad Kheirkhah, San Antonio, TX (US); Nasim Annabi, Los Angeles, CA (US); Ehsan Shirzaei Sani, Pasadena, CA (US)

(21) Appl. No.: 17/285,743

(22) PCT Filed: Oct. 16, 2019
(86) PCT No.: PCT/US2019/056521

§ 371 Date: Apr. 15, 2021

Related U.S. Application Data
(60) Provisional application No. 62/746,165, filed on Oct. 16, 2018.

(51) Int. Cl. A61L 24/04; A61L 24/00

(52) U.S. Cl. CPC A61L 24/043; A61L 24/0005; A61L 2430/16; A61L 24/0031; A61L 24/001

(57) ABSTRACT

The present invention provides compositions and methods for repair and reconstruction of defects and injuries to soft tissues. Some aspects of the invention provide tissue adhesives comprising a hybrid hydrogel by using a naturally derived polymer, gelatin and a synthetic polymer, polyethylene glycol, wherein the hydrogel is biocompatible, biodegradable, transparent, strongly adhesive to corneal tissue, and have a smooth surface and biomechanical properties similar to the cornea.

### CROSS-REFERENCE TO RELATED APPLICATIONS

This application claims benefit under 35 U.S.C. § 119(e) of U.S. Provisional Application No. 62/746,165, filed Oct. 16, 2018, the content of which is incorporated herein by reference in its entirety.

### FIELD OF THE DISCLOSURE

The field of the disclosure relates to improved tissue adhesives for use in repairing soft tissue injuries and defects.

### BACKGROUND

Corneal trauma can cause permanent visual impairment due to scar formation, neovascularization, corneal thinning, edema, or irregular astigmatism and generally accounts for nearly 5% of blindness in the world. Corneal trauma can be in different forms such as partial- or full-thickness corneal lacerations, corneal epithelial and/or stromal defects, and corneal foreign bodies. Current standards of care for major corneal lacerations have significant drawbacks. Generally, treatment options include use of cyanoacrylate glue, suture, or other types of bioadhesives. However, cyanoacrylate glue is associated with low biocompatibility, lack of transparency, rough surface, difficult handling, and lack of integration with the corneal tissue. In addition, sutures can result in regular and irregular astigmatism, neovascularization, or infection (70% of post-corneal surgery infections are suture related). Although some commercial sealants such as ReSure® (Ocular Therapeutix, Inc., USA) has been approved for sealing small corneal incisions after cataract surgery, it falls off quickly and is not designed for sealing traumatic corneal lacerations.

To allow for sutureless sealing and repair of corneal lacerations, a biocompatible and strong sealant is required which can stay on the cornea long enough for complete wound healing. Although some commercial sealants such as ReSure® (Ocular Therapeutix, Inc., USA) has been approved for sealing small corneal incisions after cataract surgery, it falls off quickly and is not designed for sealing traumatic corneal lacerations.

Because existing glues and adhesives for corneal repair have major drawbacks, there is an unmet need for an adhesive for the repair and regeneration of corneal injuries that can meet the following requirements: (1) easy application; (2) biocompatible without causing any toxicity, inflammation, or neovascularization; (3) transparent so as to enable restoration of vision as quickly as possible; (4) ability to rapidly seal the corneal wound; (5) permitting corneal cells to integrate with the bioadhesive to facilitate tissue regeneration (6) biomechanical properties (rigidity and elasticity) similar to the cornea; (7) strong adhesion to corneal tissue including good stability and high retention; and (8) smooth surface to reduce the need for bandage contact lens and minimize surface area for microbial adhesion. The present disclosure addresses some of these needs.

### SUMMARY

The inventors have developed, inter alia, a light activated bioadhesive hybrid hydrogel by using a naturally derived polymer, gelatin, and a synthetic polymer, polyethylene glycol (PEG). Gelatin and PEG are further chemically modified to form photocrosslinkable gelatin methacryloyl (GelMA) and poly(ethylene glycol) diacrylate (PEGDA). These hybrid adhesive hydrogels are biocompatible, biodegradable, transparent, strongly adhesive to corneal tissue, and have a smooth surface and biomechanical properties similar to the cornea; and are used to treat soft tissue injuries and wounds.

Certain aspects of the present invention are directed to compositions comprising acryloyl-substituted gelatin, acryloyl substituted PEG, and a visible light activated photoinitiator. In some embodiments, the visible light activated photoinitiator is used to crosslink acryloyl-substituted gelatin with acryloyl substituted PEG.

Some aspects of the invention disclose compositions comprising acryloyl-substituted gelatin cross-linked with acryloyl substituted PEG. In some embodiments of various aspects of the invention, the acryloyl-substituted gelatin cross-linked with acryloyl substituted PEG can be in form of a hydrogel.

Generally, the compositions described herein can be formulated in pharmaceutical compositions described herein. Further, these compositions can be used in methods, for eg., method to treat a soft injury or wound. Accordingly, some aspects of the invention are directed to methods for treating a soft tissue injury or wound, comprising the steps of applying acryloyl-substituted gelatin, acryloyl substituted PEG, and a visible light activated photoinitiator to the injury or wound; and applying visible light to activate the photoinitiator and cross-linking the acryloyl-substituted gelatin and the acryloyl substituted PEG.

Some aspects of the invention are directed to methods for treating a corneal defect, comprising the steps of applying acryloyl-substituted gelatin, acryloyl substituted PEG, and a visible light activated photoinitiator to the corneal defect; and applying visible light to activate the photoinitiator and cross-linking the acryloyl-substituted gelatin and the acryloyl substituted PEG.

The acryloyl-substituted gelatin can be cross-linked with acryloyl substituted PEG prior to applying to the injury or wound. Accordingly, certain aspects of the present invention are directed to method for treating a soft tissue injury or wound, comprising applying an acryloyl-substituted gelatin cross-linked with acryloyl substituted PEG to the soft tissue injury or wound. In some embodiments of various aspects of the invention, the soft tissue injury or wound is a corneal defect.

### BRIEF DESCRIPTION OF THE DRAWINGS

FIG. 1A is a schematic diagram showing design and photocrosslinking of hybrid hydrogels. The panel shows a schematic of the proposed reaction for synthesis and photocrosslinking of GelMA/PEGDA adhesive hydrogels.

FIG. 1B is a bar graph showing elastic modulus of GelMA/PEGDA adhesives. Hydrogels were produced from various polymer concentrations and 4 min visible light exposure time. Data is represented as mean±SD (*p<0.05, **p<0.01, ***p<0.001, ****p<0.0001 and n≥3).

FIG. 1C is a bar graph showing extensibility of GelMA/PEGDA adhesives. Hydrogels were produced from various polymer concentrations and 4 min visible light exposure time. Data is represented as mean±SD (*p<0.05, **p<0.01, ***p<0.001, ****p<0.0001 and n≥3).

FIG. 1D is a bar graph showing ultimate tensile strength of GelMA/PEGDA adhesives. Hydrogels were produced from various polymer concentrations and 4 min visible light exposure time. Data is represented as mean±SD (*p<0.05, **p<0.01, ***p<0.001, ****p<0.0001 and n≥3).

FIGS. 2A-2C show mechanical characterization, elastic modulus (FIG. 2A), extensibility (FIG. 2B) and ultimate tensile strength (FIG. 2C) of GelMA/PEGDA (1:1 ratio) adhesives, at different total polymer concentration. Hydrogels were formed at 4 min visible light exposure time. Data is represented as mean±SD (*p<0.05, ****p<0.0001 and n≥3). Results show that hydrogels formed with 30:30 and 50:50 GelMA/PEGDA ratios have significantly higher mechanical stability.

FIGS. 3A and 3B show rheological properties of bioadhesive prepolymer solutions. FIG. 3A shows steady-shear viscosity and FIG. 3B shows shear stress values for different of GelMA/PEGDA precusors at different PEGDA/GelMA ratio and total polymer concentration. Steady shear-viscosity results show increase of the viscosity of the prepolymer solutions, by increasing the total polymer concentration. Similar behavior was observed for shear stress values, indicating prepolymer solutions with higher concentrations require higher force to be injected.

FIGS. 4A-4F show in vitro adhesion properties of GelMA/PEGDA hydrogels using porcine skin and intestine as biological substrates. FIG. 4A is a schematic of the modified standard wound closure test (ASTM F2458-05). FIG. 4B is a bar graph showing average adhesive strength of GelMA alone and GelMA/PEGDA adhesives (n≥3) produced with varying polymer concentrations compared to commercially available adhesives, Evicel and CoSEAL. FIG. 3C is a bar graph showing adhesive strength of GelMA/PEGDA adhesives at 1:1 ratio and different total polymer concentrations (n≥3). The adhesive strength of the bioadhesives increased significantly by increasing the total polymer concentration. FIG. 4D is a schematic of the modified standard burst pressure test (ASTM F2392-04). FIG. 4E is a bar graph showing average burst pressure of GelMA/PEGDA adhesives (n≥3) produced with varying polymer concentrations compared to commercially available adhesives, Evicel and CoSEAL. FIG. 4F is a bar graph showing burst pressure values for GelMA/PEGDA adhesives at 1:1 ratio and different total polymer concentrations (n≥3). The burst pressure of the bioadhesives increased significantly by increasing the total polymer concentration, showing a maximum burst pressure at 30:30 and 50:50 GelMA/PEGDA ratios (no statistical difference). Data are means±SD (*p<0.05, **p<0.01, ***p<0.001, ****p<0.0001).

FIGS. 5A-5C show ex vivo burst pressures of visible light crosslinked GelMA and GelMA/PEGDA adhesives compared with ReSure®. FIG. 5A is a schematic showing burst pressure setup for measuring the leaking pressure of the explanted rabbit eyes with full-thickness corneal incisions of 2, 4, 6, and 8 mm in diameter, after the bioadhesives were applied and photocrosslinked. FIG. 5B is bar graph showing that the burst pressure of the corneal incisions sealed with GelMA and GelMA/PEGDA adhesives, far exceeded ReSure®. In addition, ReSure® failed to seal incisions with a diameter of 8 mm (burst pressure=0 mmHg). The crosslinking time was 4 min (***p<0.001, ****p<0.0001). FIG. 5C is a bar graph showing the burst pressure of the corneal incisions (4 mm) sealed with GelMA and GelMA/PEGDA (1:1 ratio) adhesives at different total polymer concentration. Results indicate that adhesive hydrogels formed with 30:30 and 50:50 GelMA/PEGDA ratios have remarkably higher sealing ability (burst pressure resistant) against air as compared to lower concentrations or pure GelMA. The crosslinking time was 4 min (***p<0.001, ****p<0.0001).

FIGS. 6A and 6B show ex vivo burst pressures of visible light crosslinked GelMA and GelMA/PEGDA adhesives compared with ReSure® using saline as fluid. FIG. 6A is an image of a corneal laceration on the rabbit eye after sealing with the bioadhesive hydrogel. FIG. 6B is a bar graph showing the burst pressure of the corneal incisions sealed with GelMA and GelMA/PEGDA (1:1 ratio) adhesives at different total polymer concentration used for sealing a 4 mm laceration. The crosslinking time was 4 min (****p<0.0001). Results indicate that adhesive hydrogels formed with 30:30 GelMA/PEGDA ratio has a significantly higher sealing ability (burst pressure resistant) against liquid as compared to lower concentrations, 50:50 ratio, or pure GelMA. The 50:50 GelMA/PEGDA ratio showed a lower burst pressure resistance, which is mainly due to high viscosity of the bioadhesive, causing technical difficulties for application of bioadhesive in the presence of saline solution.

### DETAILED DESCRIPTION

In one aspect, the invention provides a composition comprising acryloyl-substituted gelatin, acryloyl substituted polyethylene glycol (PEG), and a visible light activated photoinitiator. As used herein, “acryloyl-substituted gelatin” is gelatin having free amine and/or hydroxyl groups that have been substituted with at least one acryloyl group. Gelatin comprises amino acids, some of which have side chains that terminate in amines (e.g., lysine, arginine, asparagine, glutamine) or hydroxyls (e.g., serine, threonine, aspartic acid, glutamic acid). One or more of these terminal amines and/or hydroxyls can be substituted with acryloyl groups to produce acryloyl-substituted gelatin.

Gelatin is a denatured form of the connective tissue protein collagen. Several types of gelatin exist, depending on the source of collagen used, and on the extraction and production process employed. One type of gelatin is extracted from animal bones, while another type is extracted from animal skin. Usually, the animal material is from bovine or porcine origin. Depending on the extraction process, two types of gelatin can be prepared by acid hydrolysis of the collagen or by basic hydrolysis of the collagen. Both types of gelatin can be used in this invention.

Generally, an acryloyl group is an α,β-unsaturated carbonyl compound represented by the formula H₂C═CR′—C(═O)—R. As used herein, the R group is terminal amine and/or hydroxyl group on the gelatin in acryloyl substituted gelatin or gelatin derivatives. In some embodiments of different aspects of the invention, the carbon adjacent to the carbonyl carbon can be substituted with different groups (as shown in the formula as R′). Without limitations, R′ can be hydrogen, halogen, hydroxyl, C₁-C₈ alkoxy, C₁-C₈ alkyl, C₃-C₈ cycloalkyl, C₁-C₈ heteroalkyl, C₃-C₈ heterocycloalkyl, aryl, heteroaryl or amino group optionally substituted with halogen, C₁-C₈ alkoxy, C₁-C₈ alkyl, C₃-C₈ cycloalkyl, C₁-C₈ heteroalkyl, C₃-C₈ heterocycloalkyl, aryl, heteroaryl and amino group.

Exemplary halogen substituents for R′ include but are not limited to, fluorine, chlorine, bromine and iodine. Exemplary alkoxy substituents for R′, include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like. Exemplary alkyl substituents for R′ include but are not limited to, methyl, ethyl, n-propyl, isopropyl, n-butyl, isobutyl, sec-butyl, tert-butyl, pentyl, hexyl, and the like. Exemplary cycloalkyl groups for R′ include but are not limited to, optionally substituted cyclopropyl, cyclobutyl, cyclopentyl, cyclohexyl and the like. Exemplary aryl groups for R include, but are not limited to phenyl, 1-naphthyl, 2-naphthyl, biphenyl, pyridine, quinoline, furan, thiophene, pyrrole, imidazole, pyrazole, diphenylether, diphenylamine, benzophenone, and the like.

In some embodiments of various compositions and methods of the invention, R′ is methyl. In some embodiments, the acryloyl-substituted gelatin is methacryloyl-substituted gelatin (herein referred as GelMA or GELMA).

As used herein, “acryloyl gelatin” is defined as gelatin having free amines and/or free hydroxyls that have been substituted with at least one acrylamide group and/or at least one acrylate group. Gelatin comprises amino acids, some of which have side chains that terminate in amines (e.g., lysine, arginine, asparagine, glutamine) or hydroxyls (e.g., serine, threonine, aspartic acid, glutamic acid). One or more of these terminal amines and/or hydroxyls can be substituted with acryloyl groups to produce acryloyl gelatin comprising acrylamide and/or acrylate groups, respectively. In some embodiments, the gelatin may be functionalized with acryloyl groups by reacting gelatin with suitable reagents including, but not limited to, acrylic anhydride, acryloyl chloride, etc. Without limitations, it should be understood that acryloyl groups can be substituted.

“Methacryloyl gelatin” is defined as gelatin having free amines and/or free hydroxyls that have been substituted with at least one methacrylamide group and/or at least one methacrylate group. Gelatin comprises amino acids, some of which have side chains that terminate in amines (e.g., lysine, arginine, asparagine, glutamine) or hydroxyls (e.g., serine, threonine, aspartic acid, glutamic acid). One or more of these terminal amines and/or hydroxyls can be substituted with methacryloyl groups to produce methacryloyl gelatin comprising methacrylamide and/or methacrylate groups, respectively. In some embodiments, the gelatin may be functionalized with methacryloyl groups by reacting gelatin with suitable reagents including, but not limited to, methacrylic anhydride, methacryloyl chloride, 2-isocyanatoethyl methacrylate, 2-hydroxyethyl methacrylate, glycidyl methacrylate, methacrylic acid N-hydroxysuccinimide ester, allyl methacrylate, vinyl methacrylate, bis(2-methacryloyl)oxyethyl disulfide, 2-hydroxy-5-N-methacrylamidobenzoic acid, etc.

Polyethylene glycol (PEG) is a linear polymer terminated at each end with hydroxyl groups shown by the formula HO—(CH₂CH₂O)ₙ—H, where n typically ranges from approximately 10 to 2000. PEG is not toxic, does not tend to promote an immune response and is soluble in water and in many organic solvents. It is of great utility in a variety of biotechnical and pharmaceutical applications. In various aspects of the invention, the inventors have modified PEG to form acryloyl substituted PEG represented by the formula

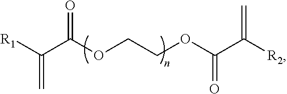

where n typically ranges from approximately 10 to 2000.

Without limitations, R₁ and R₂ can independently be hydrogen, halogen, hydroxyl, C₁-C₈ alkoxy, C₁-C₈ alkyl, C₃-C₈ cycloalkyl, C₁-C₈ heteroalkyl, C₃-C₈ heterocycloalkyl, aryl, heteroaryl or amino group optionally substituted with halogen, C₁-C₈ alkoxy, C₁-C₈ alkyl, C₃-C₈ cycloalkyl, C₁-C₈ heteroalkyl, C₃-C₈ heterocycloalkyl, aryl, heteroaryl and amino group.

It is noted that the compositions and methods of this invention contemplate using all combinations of the various substituents at R₁ and R₂. Exemplary halogen substituents for R₁ and R₂′ include but are not limited to, fluorine, chlorine, bromine and iodine. Exemplary alkoxy substituents for R₁ and R₂, include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like. Exemplary alkyl substituents for R₁ and R₂ include but are not limited to, methyl, ethyl, n-propyl, isopropyl, n-butyl, isobutyl, sec-butyl, tert-butyl, pentyl, hexyl, and the like. Exemplary cycloalkyl groups for R₁ and R₂ include but are not limited to, optionally substituted cyclopropyl, cyclobutyl, cyclopentyl, cyclohexyl and the like. Exemplary aryl groups for R₁ and R₂ include, but are not limited to phenyl, 1-naphthyl, 2-naphthyl, biphenyl, pyridine, quinoline, furan, thiophene, pyrrole, imidazole, pyrazole, diphenylether, diphenylamine, benzophenone, and the like.

In some embodiments of various compositions and methods of the invention, R₁ can be same as R₂. For example, both R₁ and R₂ can be hydrogen, methyl or ethyl. In some embodiments, R₁ and R₂ are different. For example, R₁ can be hydrogen and R₂ can be methyl. It is noted that the compositions and methods of this invention contemplate using all combinations of the various substituents at R′, R₁ and R₂.

In some embodiments of various compositions and methods of the invention, R₁ and R₂ are hydrogen. Such acryloyl substituted PEG are known as polyethylene glycol diacrylate (referred as PEGDA herein). Without limitations, the acryloyl substituted PEG of has a molecular weight between about 5 kDa to about 200 kDa. In some embodiments, the acryloyl substituted polyethylene glycol has a molecular weight between about 10 kDa to about 150 kDa. In some embodiments, the acryloyl substituted polyethylene glycol has a molecular weight between about 10 kDa to about 100 kDa. In some embodiments, the acryloyl substituted polyethylene glycol has a molecular weight between about 10 kDa to about 50 kDa. In some embodiments, the acryloyl substituted polyethylene glycol has a molecular weight between about 15 kDa to about 40 kDa. In some embodiments, the acryloyl substituted polyethylene glycol has a molecular weight between about 20 kDa to about 35 kDa.

Exemplary acryloyl substituted polyethylene glycol include, but not limited to PEGDA, polyethylene glycol monoacrylate, polyethylene glycol dimethaacrylate, polyethylene glycol monomethaacrylate, methoxy polyethylene glycol acrylate, methoxy polyethylene glycol methacrylate, ethoxy polyethylene glycol acrylate, ethoxy polyethylene glycol methacrylate, propoxy polyethylene glycol acrylate, propoxy polyethylene glycol methacrylate and the like.

For example, PEGDA has a molecular weight between about 5 kDa to about 200 kDa. In some embodiments, PEGDA has a molecular weight between about 10 kDa to about 150 kDa. In some embodiments, polyethylene glycol diacrylate has a molecular weight between about 10 kDa to about 100 kDa. In some embodiments, PEGDA has a molecular weight between about 10 kDa to about 50 kDa. In some embodiments, PEGDA has a molecular weight between about 15 kDa to about 40 kDa. In some embodiments, polyethylene glycol diacrylate has a molecular weight between about 20 kDa to about 35 kDa.

Generally, the concentration of acryloyl-substituted gelatin is defined as the weight of acryloyl-substituted gelatin divided by the volume of solvent (w/v), expressed as a percentage. The solvent may be a pharmaceutically acceptable carrier. It is also understood that the concentration can be expressed as weight/volume (w/v), mass/volume (m/v), weight/weight (w/w) or mass/mass (m/m). In some embodiments, the acryloyl-substituted gelatin is present at a concentration between 1% and 50% (w/v, m/v, w/w or m/m), between 1% and 40% (w/v, m/v, w/w or m/m), between 5% and 35% (w/v, m/v, w/w or m/m), between 10% and 30% (w/v, m/v, w/w or m/m), between 15% and 25% (w/v, m/v, w/w or m/m), or about 20% (w/v, m/v, w/w or m/m). In some embodiments, the acryloyl-substituted gelatin is present at a concentration between 5% and 15% (w/v, m/v, w/w or m/m), between 8% and 12% (w/v, m/v, w/w or m/m), or about 10% (w/v, m/v, w/w or m/m). In some embodiments, the acryloyl-substituted gelatin is present at a concentration between 10% and 40% (w/v, m/v, w/w or m/m), 15% and 35% (w/v, m/v, w/w or m/m), 20% and 30% (w/v, m/v, w/w or m/m), or about 5%, 10%, 15%, 20%, 25%, 30%, 35%, 40% or 50% (w/v, m/v, w/w or m/m).

In some embodiments of various aspects of the invention, the acryloyl-substituted gelatin is methacryloyl-substituted gelatin. The concentration of acryloyl-substituted gelatin is defined as the weight of acryloyl-substituted gelatin divided by the volume of solvent (w/v), mass/volume (m/v), weight/weight (w/w) or mass/mass (m/m) expressed as a percentage. In some embodiments, the methacryloyl-substituted gelatin is present at a concentration between 1% and 40% (w/v, m/v, w/w or m/m), between 5% and 35% (w/v, m/v, w/w or m/m), between 10% and 30% (w/v, m/v, w/w or m/m), between 15% and 25% (w/v, m/v, w/w or m/m), or about 20% (w/v, m/v, w/w or m/m). In some embodiments, the methacryloyl-substituted gelatin is present at a concentration between 5% and 15% (w/v, m/v, w/w or m/m), between 8% and 12% (w/v, m/v, w/w or m/m), or about 10% (w/v, m/v, w/w or m/m). In some embodiments, the methacryloyl-substituted gelatin is present at a concentration between 10% and 40% (w/v, m/v, w/w or m/m), 15% and 35% (w/v, m/v, w/w or m/m), 20% and 30% (w/v, m/v, w/w or m/m), or about 5%, 10%, 15%, 20%, 25%, 30%, 35%, 40% or 50% (w/v, m/v, w/w or m/m).

Generally, the concentration of acryloyl-substituted polyethylene glycol is defined as the weight of acryloyl-substituted gelatin divided by the volume of solvent (w/v), expressed as a percentage. The solvent may be a pharmaceutically acceptable carrier. It is also understood that the concentration can be expressed as weight/volume (w/v), mass/volume (m/v), weight/weight (w/w) or mass/mass (m/m). In some embodiments, the acryloyl-substituted polyethylene glycol is present at a concentration between 1% and 40% (w/v, m/v, w/w or m/m), between 5% and 35% (w/v, m/v, w/w or m/m), between 10% and 30% (w/v, m/v, w/w or m/m), between 15% and 25% (w/v, m/v, w/w or m/m), or about 20% (w/v, m/v, w/w or m/m). In some embodiments, the acryloyl-substituted polyethylene glycol is present at a concentration between 5% and 15% (w/v, m/v, w/w or m/m), between 8% and 12% (w/v, m/v, w/w or m/m), or about 10% (w/v, m/v, w/w or m/m). In some embodiments, the acryloyl-substituted polyethylene glycol is present at a concentration between 10% and 40% (w/v, m/v, w/w or m/m), 15% and 35% (w/v, m/v, w/w or m/m), 20% and 30% (w/v, m/v, w/w or m/m), or about 5%, 10%, 15%, 20%, 25%, 30%, 35%, 40% or 50% (w/v, m/v, w/w or m/m).

In some embodiments of various aspects of the invention, the acryloyl-substituted polyethylene glycol is diacrylated polyethylene glycol. The concentration of diacrylated polyethylene glycol is defined as the weight of acryloyl-substituted gelatin divided by the volume of solvent (w/v), mass/volume (m/v), weight/weight (w/w) or mass/mass (m/m) expressed as a percentage. In some embodiments, the diacrylated polyethylene glycol is present at a concentration between 1% and 40% (w/v, m/v, w/w or m/m), between 5% and 35% (w/v, m/v, w/w or m/m), between 10% and 30% (w/v, m/v, w/w or m/m), between 15% and 25% (w/v, m/v, w/w or m/m), or about 20% (w/v, m/v, w/w or m/m). In some embodiments, the diacrylated polyethylene glycol is present at a concentration between 5% and 15% (w/v, m/v, w/w or m/m), between 8% and 12% (w/v, m/v, w/w or m/m), or about 10% (w/v, m/v, w/w or m/m). In some embodiments, the PEGDA is present at a concentration between 10% and 40% (w/v, m/v, w/w or m/m), 15% and 35% (w/v, m/v, w/w or m/m), 20% and 30% (w/v, m/v, w/w or m/m), or about 5%, 10%, 15%, 20%, 25%, 30%, 35%, 40% or 50% (w/v, m/v, w/w or m/m).

Certain embodiments of the invention comprise acryloyl-substituted gelatin and acryloyl substituted polyethylene glycol in a ratio from about 30:1 to about 1:30, wherein ratio is weight to weight, mass to mass, or % (weight/volume) to % (weight/volume). In some embodiments of various aspects of the invention, acryloyl-substituted gelatin and acryloyl substituted polyethylene glycol are present in a % (weight/volume) to % (weight/volume) ratio from about 25:1 to about 1:25. For example, acryloyl-substituted gelatin and acryloyl substituted polyethylene glycol are present in a % (weight/volume) to % (weight/volume) ratio from about 2:1 to about 1:2, preferably from about 1.5:1 to about 1:1.5, more preferably about 1:1.

Certain embodiments of the invention comprise methacryloyl-substituted gelatin and diacrylated polyethylene glycol in a ratio from about 30:1 to about 1:30, wherein ratio is weight to weight, mass to mass, or % (weight/volume) to % (weight/volume). In some embodiments of various aspects of the invention, methacryloyl-substituted gelatin and diacrylated polyethylene glycol are present in a % (weight/volume) to % (weight/volume) ratio from about 25:1 to about 1:25. In some embodiments of various aspects of the invention, methacryloyl-substituted gelatin and diacrylated polyethylene glycol are present in a (weight/volume) to % (weight/volume) ratio from about 2:1 to about 1:2, preferably from about 1.5:1 to about 1:1.5, more preferably about 1:1.

As used herein, the degree of acryloyl substitution is defined as the percentage of free amines or hydroxyls in the gelatin that have been substituted with acryloyl groups. In some embodiments of various aspects of the invention, acryloyl-substituted gelatin has a degree of acryloyl substitution between 50% and 90%. Some exemplary embodiments include acryloyl-substituted gelatin having a degree of acryloyl substitution between 55% and 85%, between 60% and 80%, between 65% and 75%, between 70% and 75% or about 50%, 60%, 70%, 80% or 90%.

The degree of methacryloyl substitution is defined as the percentage of free amines or hydroxyls in the gelatin that have been substituted with methacryloyl groups. In some embodiments of various aspects of the invention, methacryloyl-substituted gelatin has a degree of methacryloyl substitution between 50% and 90%. Some exemplary embodiments include methacryloyl-substituted gelatin having a degree of methacryloyl substitution between 55% and 85%, between 60% and 80%, between 65% and 75%, between 70% and 75% or about 50%, 60%, 70%, 80% or 90%.

Certain exemplary embodiments of the present invention comprise a photoinitiator. “Photoinitiator” as used herein refers to any chemical compound, or a mixture of compounds, that decomposes into free radicals when exposed to light. Preferably, the photoinitiator produces free radicals when exposed to visible light. Exemplary ranges of visible light useful for exciting a visible light photoinitiator include green, blue, indigo, and violet. Preferably, the visible light has a wavelength in the range of 400-600 nm. Examples of photoinitiators include, but are not limited to, Eosin Y, triethanolamine, vinyl caprolactam, dl-2,3-diketo-1,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide (Ir819), 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, Diphenyl(2,4,6 trimethylbenzoyl)phosphine oxide 2-Hydroxy-2-methylpropiophenone, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane (Ivocerin®), 2-hydroxy-4′-(2-hydroxyethoxy)-2-methylpropiophenone, lithium phenyl-2,4,6-trimethylbenzoylphospinate, 2-hydroxy-2-methylpropiophenone, camphorquinone, 2-Benzyl-2-(dimethylamino)-4′-morpholinobutyrophenone, methybenzoylformate, bis(2,4,6-trimethylbenzoyl)-phenylphosphineoxide, bis(.eta.5-2,4-cylcopentadien-1-yl)-bis(2,6-difluoro-3-(1H-pyrrol-1-yl)-phenyl) titanium, 5,7-diiodo-3-butoxy-6-fluorone, 2,4,5,7-Tetraiodo-3-hydroxy-6-fluorone, 2,4,5,7-Tetraiodo-3-hydroxy-9-cyano-6-fluorone, derivatives thereof, combinations thereof, etc.

In some embodiments, the visible light activated photoinitiator is selected from the group consisting of: Eosin Y, triethanolamine, vinyl caprolactam, dl-2,3-diketo-1,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide (Ir819), 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, Diphenyl(2,4,6 trimethylbenzoyl)phosphine oxide 2-Hydroxy-2-methylpropiophenone, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane (Ivocerin®), 2-hydroxy-4′-(2-hydroxyethoxy)-2-methylpropiophenone, lithium phenyl-2,4,6-trimethylbenzoylphospinate, 2-hydroxy-2-methylpropiophenone, camphorquinone, 2-Benzyl-2-(dimethylamino)-4′-morpholinobutyrophenone, methybenzoylformate, bis(2,4,6-trimethylbenzoyl)-phenylphosphineoxide, bis(.eta.5-2,4-cylcopentadien-1-yl)-bis(2,6-difluoro-3-(1H-pyrrol-1-yl)-phenyl) titanium, 5,7-diiodo-3-butoxy-6-fluorone, 2,4,5,7-Tetraiodo-3-hydroxy-6-fluorone, 2,4,5,7-Tetraiodo-3-hydroxy-9-cyano-6-fluorone, derivatives thereof, and any combination thereof.

In some embodiments, the composition comprises at least two different photoinitiators. In some embodiments, the visible light activated photoinitiator comprises a mixture of Eosin Y, triethanolamine, and vinyl caprolactam. In some embodiments of the photoinitiator mixture, the concentration of Eosin Y is between 0.0125 and 0.5 mM, and/or the concentration of triethanolamine is between 0.1 and 2% w/v, and/or the concentration of vinyl caprolactam is between 0.05 and 1.5% w/v.

In some embodiments of the photoinitiator mixture, the concentration of Eosin Y is between 0.025 and 0.15 mM, and/or the concentration of triethanolamine is between 0.2 and 1.6% w/v, and/or and the concentration of vinyl caprolactam is between 0.09 and 0.8% w/v. In some embodiments of the photoinitiator mixture, the concentration of Eosin Y is between 0.025 and 0.15 mM, and/or the concentration of triethanolamine is between 0.2 and 1.6% w/v, and/or the concentration of vinyl caprolactam is between 0.09 and 0.8% w/v. In some embodiments of the photoinitiator mixture, the concentration of Eosin Y is between 0.05 and 0.08 mM, and/or the concentration of triethanolamine is between 0.4 and 0.8% w/v, and/or the concentration of vinyl caprolactam is between 0.18 and 0.4% w/v. In some embodiments of the photoinitiator mixture, the concentration of Eosin Y is about 0.05 mM, and/or the concentration of triethanolamine is about 0.4% w/v, and/or the concentration of vinyl caprolactam is about 0.4% w/v. In some embodiments of the photoinitiator mixture, the concentration of Eosin Y is between 0.5 and 0.5 mM, and/or the concentration of triethanolamine is between 0.5 and 2% w/v, and/or the concentration of vinyl caprolactam is between 0.5 and 1.5% w/v. In some embodiments of the photoinitiator mixture, the concentration of Eosin Y is about 0.1 mM, the concentration of triethanolamine is about 0.5% w/v, and the concentration of vinyl caprolactam is about 0.5% w/v.

Generally, a light of any suitable wavelength can be used in the method of the invention. For example, the composition can be exposed to visible light with a wavelength in the range of 400 to 600 nm. Further, exposure to light can be for any desired duration of time. For example, the composition can be exposed to visible light for a time period between 10 and 300 seconds. In some embodiments, the composition can be exposed to visible light for a time period between 20 and 120 seconds, or between 30 and 60 seconds. In some embodiments, the composition can be exposed to visible light for a time period between 60 seconds and 240 seconds. In some embodiments, the composition can be exposed to visible light for a time period of about 60 seconds, about 120 seconds, about 180 seconds or about 240 seconds. In some embodiments, the composition can be exposed to visible light for a time period of about 240 seconds.

In some embodiments of different aspects of the invention, the acryloyl-substituted gelatin, the acryloyl substituted polyethylene glycol, and the visible light activated photoinitiator are formulated in separate formulations. In some embodiments, two of the acryloyl-substituted gelatin, the acryloyl substituted polyethylene glycol, and the visible light activated photoinitiator are formulated in one formulation. In some embodiments, the acryloyl-substituted gelatin and the acryloyl substituted polyethylene glycol are formulated in one formulation. In some embodiments, all three of the acryloyl-substituted gelatin, the acryloyl substituted polyethylene glycol, and the visible light activated photoinitiator are formulated in one formulation.

In some exemplary embodiments the methacryloyl-substituted gelatin, the diacrylated polyethylene glycol, and the visible light activated photoinitiator are formulated in separate formulations. In some embodiments, two of the methacryloyl-substituted gelatin, the diacrylated polyethylene glycol, and the visible light activated photoinitiator are formulated in one formulation. In some embodiments, the methacryloyl-substituted gelatin and the diacrylated polyethylene glycol are formulated in one formulation. In some embodiments, all three of the methacryloyl-substituted gelatin, the diacrylated polyethylene glycol, and the visible light activated photoinitiator are formulated in one formulation.

In certain exemplary embodiments the methacryloyl-substituted gelatin, the diacrylated polyethylene glycol, Eosin Y, triethanolamine and vinyl caprolactam are formulated in separate formulations. In some embodiments, two of the methacryloyl-substituted gelatin, the diacrylated polyethylene glycol, Eosin Y, triethanolamine and vinyl caprolactam are formulated in one formulation. In some embodiments, the methacryloyl-substituted gelatin and the diacrylated polyethylene glycol are formulated in one formulation. In some embodiments, all of the methacryloyl-substituted gelatin, the diacrylated polyethylene glycol, Eosin Y, triethanolamine and vinyl caprolactam are formulated in one formulation.

Without limitations, with exposure to visible light in the presence of a photoinitiator, the acryloyl groups on gelatin molecule can react with the acryloyl groups on acryloyl substituted PEG molecule to crosslink the gelatin with polyethylene glycol.

Certain exemplary embodiments of the present invention comprise a pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS) or water.

In some embodiments, the composition further comprises a therapeutic agent. Exemplary therapeutic agents for inclusion in the compositions include, but are not limited to, an antibacterial, an anti-fungal, an anti-viral, an anti-acanthamoebal, an anti-inflammatory, an immunosuppressive, an anti-glaucoma, an anti-VEGF, a growth factor, or any combination thereof.

In order to promote healing and regrowth of the cornea, to prevent or treat infections or immune response, to prevent or treat corneal vessel formation, to treat increased intraocular pressure, or to promote general eye health, the compositions of the present invention may further comprise a therapeutic agent. Non-limiting examples of therapeutic agents include an antibacterial, an anti-fungal, an anti-viral, an anti-acanthamoebal, an anti-inflammatory, an immunosuppressive, an anti-glaucoma, an anti-VEGF, a growth factor, or any combination thereof. Non-limiting examples of antibacterial agents include: penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim sulfamethoxazole, chitosan, ansamycins, daptomycin, nitrofurans, oxazolidinones, bacitracin, colistin, polymixin B, and clindamycin. Non-limiting examples of anti-fungal agents include: amphotericin B, natamycin, candicin, filipin, hamycin, nystatin, rimocidin, voriconazole, imidazoles, triazoles, thiazoles, allylamines, echinocandins, benzoic acid, ciclopirox, flucytosine, griseofulvin, haloprogin, tolnaftate, undecylenic acid, and povidone-iodine. Non-limiting examples of anti-viral agents include: acyclovir, valacyclovir, famciclovir, penciclovir, trifluridine, and vidarabine. Non-limiting examples of anti-acanthamoebal agents include: chlorohexidine, polyhexamethylen biguanide, propamidine, and hexamidine. Non-limiting examples of anti-inflammatory agents include: corticosteroids; non-steroidal anti-inflammatory drugs including salicylates, propionic acid derivatives, acetic acid derivatives, enolic acid derivatives, anthranilic acid derivatives, selective cox-2 inhibitors, and sulfonanilides; biologicals including antibodies (such as tumor necrosis factor-alpha inhibitors) and dominant negative ligands (such as interleukin-1 receptor antagonists). Non-limiting examples of immunosuppressive agents include: alkylating agents, antimetabolites, mycophenolate, cyclosporine, tacrolimus, and rapamycin. Non-limiting examples of anti-glaucoma agents include: prostaglandin analogs, beta blockers, adrenergic agonists, carbonic anhydrase inhibitors, parasympathomimetic (miotic) agents. Non-limiting examples of anti-vascular endothelial growth factor (anti-VEGF) agents include: bevacizumab, ranibizumab, and aflibercept. Non-limiting examples of growth factors include: epidermal growth factor, platelet-derived growth factor, vitamin A, fibronectin, annexin a5, albumin, alpha-2 macroglobulin, fibroblast growth factor b, insulin-like growth factor-I, nerve growth factor, and hepatocyte growth factor.

Without limitations, the compositions and methods described herein can further comprise a cell. Generally, any type of cells can be used but not limited to corneal cells, endothelial cells, skin cells, nerve cells, bone cells, muscle cells, blood cells, stem cells etc.

In some embodiments, the composition further comprises corneal cells. Exemplary, corneal cells include, but are not limited to, epithelial cells, endothelial cells, keratocytes, and any combinations thereof.

Corneal cells may be incorporated in or on the surface of the bioadhesive in order to promote corneal tissue formation and healing. Thus, in some embodiments, the GelMA composition further comprises corneal cells, preferably epithelial cells, endothelial cells, keratocytes, or a combination thereof. Epithelial and/or endothelial cells are preferably seeded on the surface of the composition, while keratocytes are preferably mixed into the composition prior to photopolymerization.

The compositions described herein can be administered by any appropriate route known in the art including, but not limited to, oral or parenteral routes, including intravenous, topical, intramuscular, subcutaneous, transdermal, airway (aerosol), pulmonary, nasal and rectal administration. In some embodiments, the composition is formulated for topical administration.

The inventors have developed, inter alia, a novel bioadhesive hybrid hydrogel by using a naturally derived polymer, gelatin, and a synthetic polymer, polyethylene glycol (PEG). Gelatin and PEG are further chemically modified to form photocrosslinkable GelMA and PEGDA. Different ratios of GelMA and PEGDA can be photocrosslinked in the presence of a photoinitiator upon short-time exposure to visible light (400-600 nm), forming solid hydrogels that firmly adhere to the corneal tissue. Physical and chemical properties of the resulting hydrogels can be finely tuned so that they can be used for different surgical and tissue engineering applications, particularly for corneal repair. These tissue adhesives hybrid hydrogels are biocompatible, biodegradable, transparent, strongly adhesive to corneal tissue, and have a smooth surface and biomechanical properties similar to the cornea.

Certain aspects of the present invention are directed to compositions comprising acryloyl-substituted gelatin crosslinked with acryloyl substituted PEG. These compositions are also referred to as cross-linked compositions herein. In some embodiments, methacryloyl-substituted gelatin is crosslinked with PEGDA. As used herein, polyethylene glycol diacrylate and diacrylated polyethylene glycol have been used interchangeably. In some embodiments, the compositions are in the form of a hydrogel.

Certain aspects of the present invention are directed to a composition for corneal reconstruction comprising a crosslinked methacryloyl-substituted gelatin hydrogel and a pharmaceutically acceptable carrier. As used herein, a “hydrogel” is a network of hydrophilic polymer chains forming a colloidal gel. In some embodiments, the crosslinked methacryloyl-substituted gelatin hydrogel has a degree of methacryloyl substitution between 50% and 90%.

Although widespread in biomedical applications, UV light crosslinking has potential biosafety concerns as it may lead to undesired DNA damage and ocular toxicity. Methacryloyl substituted gelatin comprises modified natural extracellular matrix components that can be crosslinked with acryloyl substituted polyethylene glycol via visible light exposure to create an elastic and biodegradable hydrogel for corneal reconstruction and repair. Natural extracellular matrix components may include gelatin derived from animals including, but not limited to, pig, cow, horse, chicken, fish, etc. Advantageously, the gelatin can be harvested under sterile conditions from animals in pathogen-free barrier facilities to eliminate the risk of transmission of disease (e.g, hepatitis C, human immunodeficiency virus, etc.)

In situ photopolymerization of methacryloyl substituted gelatin with PEGDA facilitates easy delivery to technically demanding locations such as the cornea, and allows for curing of the bioadhesive exactly according to the required geometry of the tissue to be sealed, which is an advantage over pre-formed materials, as e.g., scaffolds or sheets.

As used herein, “methacryloyl gelatin” is defined as gelatin having free amines and/or free hydroxyls that have been substituted with at least one methacrylamide group and/or at least one methacrylate group. Gelatin comprises amino acids, some of which have side chains that terminate in amines (e.g., lysine, arginine, asparagine, glutamine) or hydroxyls (e.g., serine, threonine, aspartic acid, glutamic acid). One or more of these terminal amines and/or hydroxyls can be substituted with methacryloyl groups to produce methacryloyl gelatin comprising methacrylamide and/or methacrylate groups, respectively. In some embodiments, with exposure to visible light in the presence of a photoinitiator, the methacryloyl groups on gelatin molecule can react with the polyethylene glycol diacrylate to crosslink and produce a hydrogel. In some embodiments, the gelatin may be functionalized with methacryloyl groups by reacting gelatin with suitable reagents including, but not limited to, methacrylic anhydride, methacryloyl chloride, 2-isocyanatoethyl methacrylate, 2-hydroxyethyl methacrylate, glycidyl methacrylate, methacrylic acid N-hydroxysuccinimide ester, allyl methacrylate, vinyl methacrylate, bis(2-methacryloyl)oxyethyl disulfide, 2-hydroxy-5-N-methacrylamidobenzoic acid, etc.

The mechanical properties of the hydrogel can be tuned for various applications by changing the degree of methacryloyl substitution, concentration of methacryloyl substituted gelatin, concentration of polyethylene glycol diacrylate, amount of photoinitiators, and light exposure time.

The physical properties (degradation and mechanical properties, etc.) of the hydrogel can be modified so that different compositions of the bioadhesive can be made for different purposes, e.g., a bioadhesive with either short or long retention time, appropriate for different clinical scenarios. For example, in the case of a corneal trauma with extruded intraocular contents such as iris, one may wish to apply hydrogel for temporary sealing of the injured eye. In patients with corneal epithelial defects, hydrogel with short retention time may also be used to cover the epithelial defect. In contrast, in the case of a cornea with a structural defect or severe thinning, hydrogel can be formulated in a way that it retains for prolonged periods. Currently available sealant technologies (e.g. cyanoacrylate) do not offer such control in the characteristics of the final product.

The following are desired physical properties, either alone or in combination, for bioadhesive compositions suitable for corneal repair. In some embodiments, the cross-linked acryloyl-substituted gelatin has an extensibility of 20-100%, between 30-90%, between 40-80%, between 50-70%, or 20%, 30%, 40%, 50%, 60%, 70%, 80%, 90% or 100%. In some embodiments, the cross-linked acryloyl-substituted gelatin has an elastic modulus of 5-150 kPa, between 10-130 kPa, between 20-100 kPa, between 30-80 kPa, between 40-70 kPa or between 50-60 kPa. In some embodiments, the cross-linked acryloyl-substituted gelatin has an ultimate stress of 5-40 kPa, between 10-35 kPa, between 15-30 kPa or between 20-25 kPa. In some embodiments, the cross-linked acryloyl-substituted gelatin has an adhesion strength of 20-90 kPa, between 30-70 kPa, between 40-60 kPa or between 45-55 kPa. In some embodiments, the cross-linked acryloyl-substituted gelatin has an adhesion strength between 37.2±5.3 kPa and 78.1±7.84 kPa. In some embodiments, the cross-linked acryloyl-substituted gelatin has burst pressure of ≥20 kPa. In some embodiments, the cross-linked acryloyl-substituted gelatin has burst pressure between 30-35 kPa. In some embodiments, the cross-linked acryloyl-substituted gelatin has burst pressure of 30.1±4.3 kPa.

In some embodiments, the composition is substantially clear. In some embodiments, the composition has a substantially smooth surface.

Some aspects of the invention are directed to methods for treating a soft tissue injury or wound, comprising the steps of applying acryloyl-substituted gelatin, acryloyl substituted polyethylene glycol, and a visible light activated photoinitiator to the injury or wound; and applying visible light to activate the photoinitiator and cross-linking the acryloyl-substituted gelatin and the acryloyl substituted polyethylene glycol.

Generally, soft tissue includes all tissue of the body except bone. Examples of soft tissue include, but are not limited to, muscles, tendons, fibrous tissues, fat, blood vessels, nerves, and synovial tissues. As used herein, the term “wound” is used to describe skin wounds as well as tissue wounds. A skin wound is defined herein as a break in the continuity of skin tissue that is caused by direct injury to the skin. Several classes including punctures, incisions, excisions, lacerations, abrasions, atrophic skin, or necrotic wounds and burns generally characterize skin wounds. In some embodiments, the compositions and methods of the invention are useful for enhancing the healing of wounds of the skin, cornea, heart, liver, cartilage, bones, vascular system, spleen, kidney, stomach and intestinal wounds.

In some preferred embodiments, the wound is a cornea, heart, liver, spleen, kidney, stomach and intestinal wound. In yet another preferred embodiment, the soft tissue injury or wound is a corneal defect.

Some aspects of the invention are directed to methods for treating a corneal defect, comprising the steps of applying acryloyl-substituted gelatin, acryloyl substituted polyethylene glycol, and a visible light activated photoinitiator to the corneal defect; and applying visible light to activate the photoinitiator and cross-linking the acryloyl-substituted gelatin and the acryloyl substituted polyethylene glycol.

Certain exemplary aspects of the invention are directed to methods for treating a corneal defect, comprising the steps of applying methacryloyl-substituted gelatin, polyethylene glycol diacrylate, Eosin Y, vinyl caprolactam and triethanolamine to the corneal defect; and applying visible light to activate the photoinitiator and cross-linking the acryloyl-substituted gelatin and the acryloyl substituted polyethylene glycol.

The acryloyl-substituted gelatin can be cross-linked with acryloyl substituted polyethylene glycol prior to applying to the injury or wound. Accordingly, certain aspects of the present invention are directed to method for treating a soft tissue injury or wound, comprising applying an acryloyl-substituted gelatin cross-linked with acryloyl substituted polyethylene glycol to the soft tissue injury or wound. In some embodiments of various aspects of the invention, the soft tissue injury or wound is a corneal defect.

The mechanical properties of the hydrogel can be tuned for various applications by changing the visible light exposure time. Without being bound by theory, longer visible light exposure time produces more crosslinkage in the methacryloyl-substituted gelatin, providing a hydrogel with improved mechanical properties, such as adhesion strength, shear strength, compressive strength, tensile strength, etc. In some embodiments, the composition is exposed to visible light for a time period between 30 seconds and 6 minutes, between 1 minute and 5 minutes, between 2 minutes and 4 minutes, or 3 minutes. In some embodiments, the composition is exposed to visible light for a time period of less than one minute, within 10-60 seconds, 15-45 seconds, 20 seconds, or 30 seconds. In some embodiments, the composition is exposed to visible light for a time period between 20 and 120 seconds, or between 30 and 60 seconds. In some embodiments, the composition can be exposed to visible light for a time period between 60 seconds and 240 seconds. In some embodiments, the composition can be exposed to visible light for a time period of about 60 seconds, about 120 seconds, about 180 seconds or about 240 seconds.

In some embodiments, the method does not comprise suturing the cornea. Exemplary ranges of visible light useful for crosslinking the compositions described herein include green, blue, indigo, and violet. Preferably, the visible light has a wavelength in the range of 400-600 nm.

Some embodiments of the technology described herein can be defined according to any of the following numbered paragraphs:
1. A composition comprising acryloyl-substituted gelatin, acryloyl substituted polyethylene glycol (PEG), and a visible light activated photoinitiator.
2. The composition of paragraph 1, wherein the composition further comprises a pharmaceutically acceptable carrier or excipient.
3. The composition of paragraph 1 or 2, wherein the composition comprises acryloyl-substituted gelatin in an amount from about 1% to about 40%, wherein the weight % is weight/volume, mass/volume, weight/weight or mass/mass.
4. The composition of any one of paragraphs 1-3, wherein composition comprises acryloyl substituted polyethylene glycol in an amount from about 1% to about 40%, wherein the % is weight/volume, mass/volume, weight/weight or mass/mass.
5. The composition of any one of paragraphs 1-4, wherein the acryloyl-substituted gelatin, acryloyl substituted polyethylene glycol are present in a ratio from about 30:1 to about 1:30, wherein ratio is weight to weight, mass to mass, or % (w/v) to % (w/v).
6. The composition of any one of paragraphs 1-5, wherein the acryloyl-substituted gelatin, acryloyl substituted polyethylene glycol are present in a % (w/v) to % (w/v) ratio from about 25:1 to about 1:25.
7. The composition of any one of paragraphs 1-6, wherein the acryloyl-substituted gelatin is methacryloyl-substituted gelatin.
8. The composition of any one of paragraphs 1-7, wherein acryloyl-substituted gelatin has a degree of acryloyl substitution between 50% and 90%.
9. The composition any one of paragraphs 1-8, wherein the acryloyl substituted polyethylene glycol is diacrylated polyethylene glycol (PEGDA).
10. The composition of any one of paragraphs 1-9, wherein the acryloyl substituted polyethylene glycol has a molecular weight between about 5 kDa to about 200 kDa.
11. The composition of any one of paragraphs 1-10, wherein the composition comprises at least two different photoinitiators.
12. The composition of any one of paragraphs 1-11, wherein composition further comprises a therapeutic agent.
13. The composition of any one of paragraphs 1-12, wherein the composition further comprises a cell.
14. The composition of any one of paragraphs 1-13, wherein the cell is a corneal cell.
15. The composition of any one of paragraphs 1-14, wherein the composition is formulated for topical use.
16. A composition comprising acryloyl-substituted gelatin cross-linked with acryloyl substituted polyethylene glycol.
17. The composition of paragraph 16, wherein the composition is in form of a hydrogel.
18. The composition of paragraph 16 or 17, wherein the composition further comprises a pharmaceutically acceptable carrier or excipient.
19. The composition of any one of paragraphs 16-18, wherein the composition comprises acryloyl-substituted gelatin in an amount from about 1% to about 40%, wherein the % is weight/volume, mass/volume, weight/weight or mass/mass.
20. The composition of any one of paragraphs 16-19, wherein composition comprises acryloyl substituted polyethylene glycol in an amount from about 1% to about 40%, wherein the weight % weight/volume, mass/volume, weight/weight or mass/mass.
21. The composition of any one of paragraphs 16-20, wherein the acryloyl-substituted gelatin, acryloyl substituted polyethylene glycol are present in a ratio from about 30:1 to about 1:30, wherein ratio is weight to weight, mass to mass, or % (w/v) to % (w/v).
22. The composition of any one of paragraphs 16-21, wherein the acryloyl-substituted gelatin, acryloyl substituted polyethylene glycol are present in a % (w/v) to % (w/v) ratio from about 25:1 to about 1:25.
23. The composition of any one of paragraphs 16-22, wherein the acryloyl-substituted gelatin is methacryloyl-substituted gelatin.
24. The composition of any one of paragraphs 16-23, wherein acryloyl-substituted gelatin has a degree of acryloyl substitution between 50% and 90%.
25. The composition any one of paragraphs 16-24, wherein the acryloyl substituted polyethylene glycol) is diacrylated polyethylene glycol.
26. The composition of any one of paragraphs 16-25, wherein the acryloyl substituted polyethylene glycol has a molecular weight between about 5 kDa to about 200 kDa.
27. The composition of any one of paragraphs 16-26, wherein the cross-linked acryloyl-substituted gelatin has an extensibility of 20-100%.
28. The composition of any one of paragraphs 16-27, wherein the cross-linked acryloyl-substituted gelatin has an elastic modulus of 5-150 kPa.
29. The composition of any one of paragraphs 16-28, wherein the cross-linked acryloyl-substituted gelatin has an ultimate stress of 5-40 kPa.
30. The composition of any one of paragraphs 16-29, wherein the cross-linked acryloyl-substituted gelatin has an adhesion strength of 20-90 kPa.
31. The composition of any one of paragraphs 16-30, wherein the cross-linked acryloyl-substituted gelatin has burst pressure of ≥20 kPa.
32. The composition of any one of paragraphs 26-31, wherein the composition is substantially clear.
33. The composition of any one of paragraphs 26-32, wherein the composition has a substantially smooth surface.
34. The composition of any one of paragraphs 16-33, wherein composition further comprises a therapeutic agent.
35. The composition of any one of paragraphs 16-34, wherein the composition further comprises a cell. 36 The composition of any one of paragraphs 16-35, wherein the cell is a corneal cell.
37. The composition of any one of paragraphs 1-14, wherein the composition is formulated for topical use.
38. A method for treating a soft tissue injury or wound, comprising:
   a. applying acryloyl-substituted gelatin, acryloyl substituted polyethylene glycol, and a visible light activated photoinitiator to the injury or wound; and
   b. applying visible light to activate the photoinitiator and cross-linking the acryloyl-substituted gelatin and the acryloyl substituted PEG.
39. The method of paragraph 38, wherein the acryloyl-substituted gelatin is applied in a composition having acryloyl-substituted gelatin in an amount from about 1% to about 40%, wherein the % is weight/volume, mass/volume, weight/weight or mass/mass.
40. The method of paragraph 38 or 39, wherein acryloyl-substituted PEG is applied in a composition having acryloyl-substituted PEG in an amount from about 1% to about 40%, wherein the weight % weight/volume, mass/volume, weight/weight or mass/mass.
41. The method of any one of paragraphs 38-40, wherein the acryloyl-substituted gelatin and the acryloyl-substituted polyethylene glycol are applied in a ratio from about 30:1 to about 1:30, wherein ratio is weight to weight, mass to mass, or % (w/v) to % (w/v).
42. The method of any one of paragraphs 38-41, wherein the acryloyl-substituted gelatin and the acryloyl-substituted polyethylene glycol are applied in a % (w/v) to % (w/v) ratio from about 25:1 to about 1:25.
43. The method of any one of paragraphs 38-42, wherein the acryloyl-substituted gelatin is methacryloyl-substituted gelatin.
44. The method of any one of paragraphs 38-43, wherein acryloyl-substituted gelatin has a degree of acryloyl substitution between 50% and 90%.
45. The method of any one of paragraphs 38-44, wherein the acryloyl substituted polyethylene glycol is diacrylated polyethylene glycol.
46. The method of any one of paragraphs 38-46, wherein the acryloyl substituted polyethylene glycol has a molecular weight between about 5 kDa to about 200 kDa.
47. The method of any one of paragraphs 38-46, wherein the visible light activated photoinitiator is a mixture of two or more different photoinitiators.
48. The method of any one of paragraphs 38-47, wherein the acryloyl-substituted gelatin, the acryloyl substituted polyethylene glycol, and the visible light activated photoinitiator are formulated in separate formulations.
49. The method of any one of paragraphs 38-47, wherein two of the acryloyl-substituted gelatin, the acryloyl substituted polyethylene glycol, and the visible light activated photoinitiator are formulated in one formulation.
50. The method of paragraph 49, wherein the acryloyl-substituted gelatin and the acryloyl substituted polyethylene glycol are formulated in one formulation.
51. The method of any one of paragraphs 38-47, wherein all three of the acryloyl-substituted gelatin, the acryloyl substituted polyethylene glycol, and the visible light activated photoinitiator are formulated in one formulation.
52. A method for treating a soft tissue injury or wound, comprising:
   a. applying a composition of any one of paragraphs 16-27 to the injury or wound; and
   b. applying visible light to activate the photoinitiator and cross-linking the acryloyl-substituted gelatin and the acryloyl substituted PEG
53. The method of any one of paragraphs 38-52, wherein the soft tissue injury or wound is selected from the group consisting of muscles, tendons, ligaments, fascia, nerves, fibrous tissues, fat, blood vessels, synovial membranes, liver, spleen, kidney, stomach and intestinal wounds.
54. The method of any one of paragraphs 38-53, wherein the soft tissue injury or wound is a corneal defect.
55. The method of any one of paragraphs 38-54, further comprising administering a therapeutic agent to the soft tissue injury or wound.
56. The method of any one of paragraphs 38-54, wherein the method does not comprise a step of suturing.

### Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%, ±1.5%, ±2%, ±2.5%, ±3%, ±3.5%, ±4%, ±4.5%, or ±5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

As used herein, the term “hydrogel” refers to a three-dimensional polymeric structure that is insoluble or minimally soluble in water or some other liquid but which is capable of absorbing and retaining large quantities of water or some other liquid to form a stable, often soft and pliable, structure.

As used herein, the term “biodegradable” describes a material which can decompose partially or fully under physiological conditions into breakdown products. The material under physiological conditions can undergo reactions or interactions such as hydrolysis (decomposition via hydrolytic cleavage), enzymatic catalysis (enzymatic degradation), and mechanical interactions. As used herein, the term “biodegradable” also encompasses the term “bioresorbable,” which describes a substance that decomposes under physiological conditions, breaking down to products that undergo bioresorption into the host-organism, namely, become metabolites of the biochemical systems of the host organism. For example, a material is biodegradable if at least 10%, at least 20%, at least 30%, at least 40%, or more preferably, at least 50%, at least 60%, at least 70%, at least 80%, at least 90% of the material can decompose under physiological conditions within a desired period of time, such as on the order of minutes, hours, days, weeks, or months, depending on the exact material.

As used herein, the term “scaffold” refers to tissue patch for wide range of biomedical applications, including eye, skin, heart, liver, cartilage, tendon, intestine, bones, vascular system, spleen, kidney, stomach and intestine, and can be attached to the tissue through its prepolymer form, without the need for any adhesive or suture.

As used herein, the term “physiological conditions” refer to conditions of temperature, pH, osmotic pressure, osmolality, oxidation and electrolyte concentration in vivo in a human patient or mammalian subject at the site of administration, or the site of action. For example, physiological conditions generally mean pH at about 6 to 8 and temperature of about 37° C. in the presence of serum or other body fluids.

As used herein, the term “biocompatible” denotes being biologically compatible by not producing a toxic, injurious, or immunological response in living tissue.

As used herein, “bioadhesive” is natural polymeric material that can act as adhesive. Bioadhesives are generally useful for biomedical applications involving skin, cornea or other soft tissue. The bioadhesive described in the invention comprise gelatin functionalized with glycidyl methacrylate.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, rabbits, deer, bison, buffalo, goats, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “individual,” “patient,” “subject,” and the like are used interchangeably herein. The terms do not denote a particular age, and thus encompass adults, children, and newborns. A subject can be a male or female.

As used herein, the term “administer” refers to the placement of a composition into a subject by a method or route which results in at least partial localization of the composition at a desired site such that desired effect is produced.

Preferably, the subject is a mammal. The mammal can be a human, non-human primate, mouse, rat, dog, cat, horse, or cow, but is not limited to these examples. Mammals other than humans can be advantageously used as subjects in animal models of human treatment or disease. In addition, the methods and compositions described herein can be used for treatment of domesticated animals and/or pets. A human subject can be of any age, gender, race or ethnic group. In some embodiments, the subject can be a patient or other subject in a clinical setting. In some embodiments, the subject can already be undergoing treatment.

As used herein, the terms “treat,” “treatment,” “treating”, or “amelioration” are used herein to characterize a method or process that is aimed at (1) delaying or preventing the onset of a disease or condition; (2) slowing down or stopping the progression, aggravation, or deterioration of the symptoms of the disease or condition; or (3) bringing about ameliorations of the symptoms of the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment). A treatment can be administered prior to the onset of the disease, for a prophylactic or preventive action. Alternatively, or additionally, the treatment can be administered after initiation of the disease or condition, for a therapeutic action.

As used herein, the term “soft tissue” includes all tissue of the body except bone. Examples of soft tissue include, but are not limited to, muscles, tendons, fibrous tissues, fat, blood vessels, nerves, and synovial tissues.

As used herein, the term “wound” is used to describe skin wounds as well as tissue wounds. A skin wound is defined herein as a break in the continuity of skin tissue that is caused by direct injury to the skin. Several classes including punctures, incisions, excisions, lacerations, abrasions, atrophic skin, or necrotic wounds and burns generally characterize skin wounds. In some embodiments, the compositions and methods of the invention are useful for enhancing the healing of wounds of the skin, cornea, heart, liver, cartilage, bones, vascular system, spleen, kidney, stomach and intestinal wounds. The terms “injury”, “wound” and “defect” have been used interchangeably herein.

The terms “bioactive agent” and “biologically active agent” are used herein interchangeably. They refer to compounds or entities that alter, inhibit, activate or otherwise affect biological events.

The term “cross-link” refers to a bond that links one polymer to another. These links can be covalent bond or ionic bonds and the polymers can be either synthetic polymers or natural polymers. When a synthetic polymer is cross-linked, the entire bulk of the polymer has been exposed to the cross-linking method.

The term “crosslinking” is process of forming covalent bonds or relatively short sequences of chemical bonds to join two polymer chains together.

It is noted that physical and chemical properties of the resulting hydrogels comprising acryloyl-substituted gelatin cross-linked with acryloyl substituted polyethylene glycol can be finely tuned so that they can be used for different surgical and tissue engineering applications, particularly for corneal repair. In particular, the formulation of the bioadhesive was modified to obtain high adhesion to the native cornea, while retaining appropriate biodegradability and high cytocompatibility in vitro. The adhesion properties of the engineered hydrogel adhesives were tested based on standard adhesion tests by the American Society for Testing and Materials (ASTM) tests and were compared to commercially available adhesives used for cornea sealing such as ReSure®. In addition, ex vivo tests on explanted rabbit eyes were performed to evaluate the retention and burst pressure resistance of the engineered bioadhesives. In vivo testing of the bioadhesive formulation using full thickness corneal laceration model in rabbits is also carried out. Advantageously, the bioadhesives of the present invention are low cost, easy to produce, and easy to use, making them a promising substance to be used for corneal repair, as well as an easily tunable platform to further optimize the adhesive characteristics.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

### EXAMPLES

The disclosure is further illustrated by the following examples which should not be construed as limiting. The examples are illustrative only, and are not intended to limit, in any manner, any of the aspects described herein. The following examples do not in any way limit the invention.

### Example 1: GelMA/PEGDA Adhesive Hybrid Hydrogel for Sealing Full Thickness Corneal Laceration

To address the limitations of current standard of care for treatment of corneal lacerations, we developed a novel bioadhesvie hybrid hydrogel by using a naturally derived polymer, gelatin, and a synthetic biopolymer, polyethylene glycol (PEG). We further chemically modified gelatin and PEG to form photocrosslinkable gelatin methacryloyl (GelMA) and Poly(ethylene glycol) diacrylate (PEGDA). By combination of GelMA and PEGDA at different ratios, in the presence of photoinitiator solution, and can be photocrosslinked upon short-time exposure to visible light (450-550 nm), forming a solid hydrogel that firmly adheres to the corneal tissue. Physical and chemical properties can be finely tuned so that it can be used for different surgical and tissue engineering applications, particularly for corneal repair. In addition, the formulation of the adhesive was modified to obtain high adhesion to the native tissue, while retaining appropriate biodegradability and high cytocompatibility in vitro. Next, the adhesion properties of the engineered hydrogel adhesives were tested based on standard adhesion tests by the American Society for Testing and Materials (ASTM) tests and were compared to commercially available adhesives used for cornea such as ReSure®. In addition, ex vivo tests on explanted rabbit eyes were performed to evaluate the retention and burst pressure resistance. Furthermore, in vivo tests were conducted using a rabbit stromal cornea defect model to test the biocompatibility and retention of the biomaterial, as well as sealing corneal laceration after the application

### Materials and Methods

Synthesis of PEGDA: To synthesize PEGDA, poly(ethylene glycol) (PEG, Sigma Aldrich) was chemically reacted with acryloyl chloride (Sigma Aldrich). Accordingly, 10 grams of PEG was dissolved in 100 ml of dichloromethane (10% w/v) at 4° C. Next, triethylamine (Sigma Aldrich) was added to the PEG solution under N₂ environment. Acryloryl chloride (Sigma Aldrich) was then added to the solution and were dissolved in the PEG solution and stirred overnight under dry N₂ gas. The molar ratio of PEG, acryloyl chloride and triethylamine was 1:4:4. Finally, the insoluble salt (triethylamine-HCl) was filtered (using celite 545 powder and alumina column), and the product was precipitated by adding ice-cold ether. The crude product was filtered with 9 μm paper filter and dried in vacuum desiccator overnight to remove unreacted materials.

Synthesis of GelMA: GelMA with 70% degree of substitution was synthesized based on the reported procedure (E. S. Sani et al., Sutureless repair of corneal injuries using naturally derived bioadhesive hydrogels, Science Advances 5 (2019) eaav1281 and E. S. Sani et al. An Antimicrobial Dental Light Curable Bioadhesive Hydrogel for Treatment of Peri-Implant Diseases, (2019). Briefly, 10% (w/v) gelatin from porcine skin (Sigma) solution in DPBS was reacted with 8 mL of methacrylic anhydride for 3 h. The solution was then dialyzed for 5 days to remove any unreacted methacrylic anhydride, and then placed in a −80° C. freezer for 24 h. The frozen polymer was then freeze-dried for 5 days.

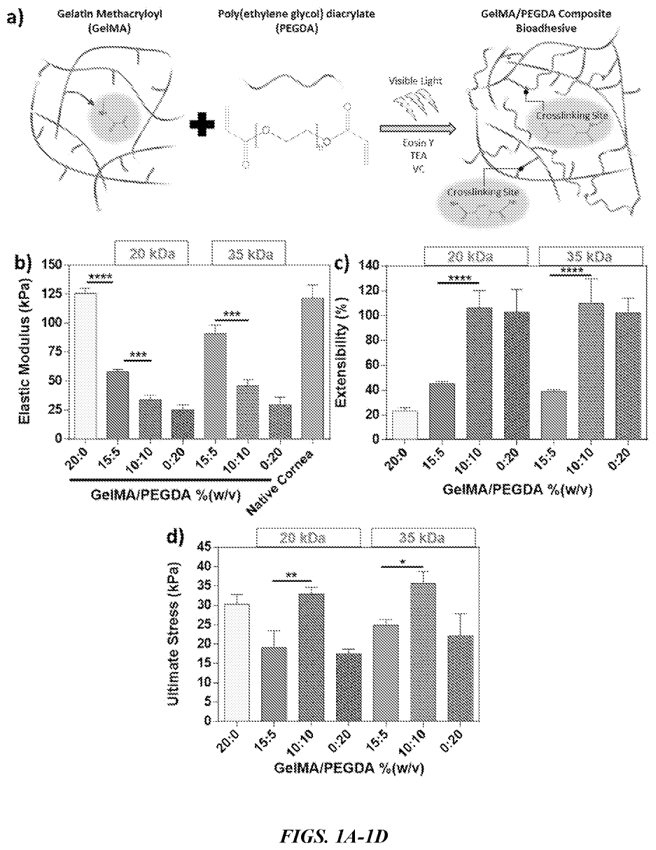

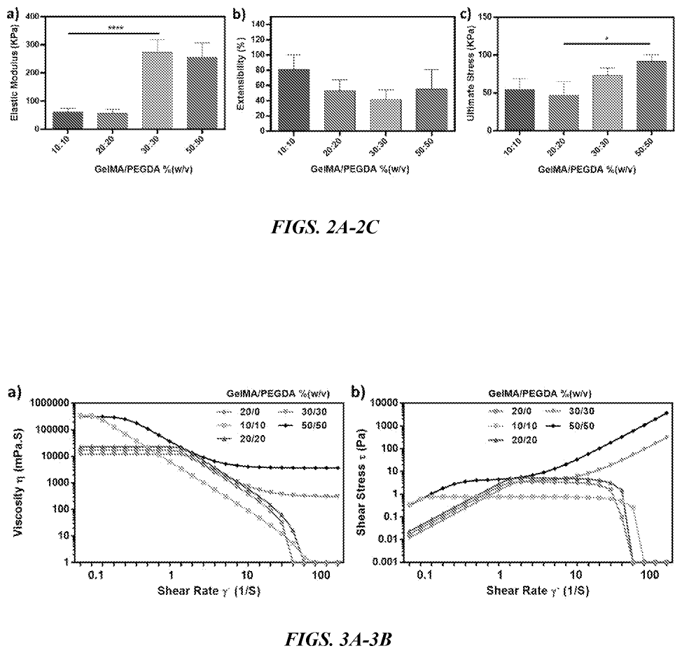

Preparation of the bioadhesive composite hydrogels: To prepare GelMA/PEGDA adhesive prepolymer solutions, the lyophilized GelMA and PEGDA were mixed in different ratios and dissolved in a solution containing triethanolamine (TEA) (1.8% w/v) and poly(N-vinylcaprolactam) (VC) (1.25% w/v) in distilled water. Eosin Y disodium salt (0.5 mM) was also dissolved separately in distilled water and added with final concentration of 0.1 Mm to the biopolymers/TEA/VC solution prior to photocrosslinking. The hydrogels were formed by exposing to visible light (400-600 nm, using a LS1000 FocalSeal Xenon Light Source (Genzyme)) for 4 min (FIG. 1A).

Mechanical characterization of the adhesive hydrogels: For compression and tensile test, the biopolymers/TEA/VC solution was mixed with Eosin Y, and 70 mL of the final solution was placed into polydimethylsiloxane (PDMS) cylindrical (diameter: 6 mm; height: 2.5 mm) molds for compressive tests, or rectangular (14×5×1 mm) molds for tensile tests. The resulting solution was photocrosslinked via exposure to visible light (480-520 nm) for 240 s. After photocrosslinking, the dimensions of the hydrogels were measured using digital calipers. Both compression and tensile tests were conducted using an Instron 5542 mechanical tester. For tensile test, the hydrogels were placed between two pieces of double sided tape within the instrument tension grips and extended at a rate of 1 mm/min until failure. The slope of the stress-strain curves was obtained and reported as elastic modulus.

For the rheological tests, different concentrations of bioadhesive precursor loaded between the parallel plates of an Anton-Paar 302 Rheometer. Steady shear viscosity assessment (frequency range: 0.01-100 rad/s) were performed at a low strain of 1.0% for the solutions at 37° C. Steady shear rate sweeps were conducted by varying the shear rate from 0.01 to 500 s⁻¹ to determine the yield stress of the prepolymer solutions.

In vitro burst pressure test: Burst pressure resistance of composite hydrogels was calculated by using the ASTM F2392-04 standard according to previously reported method (N. Annabi et al., Engineering a highly elastic human protein-based sealant for surgical applications, Science translational medicine 2017, 9(410) eaai7466). Briefly, porcine intestine (4×4 cm) was placed in between two stainless steel annuli from a custom-built burst pressure device, which consists of a metallic base holder, pressure meter, syringe pressure setup, and data collector. A hole (1 mm diameter) was created through the intestine and was sealed by applying the adhesive gels. Airflow was terminated post hydrogel rupture and the burst pressure resistant was measured using a wireless pressure sensor connected to a computer (n≥5).

In vitro wound closure test: The adhesion strength of GelMA/PEGDA adhesives with different ratios was calculated by using the ASTM F2458-05 standard according to reported procedure (N. Annabi et al., Engineering a highly elastic human protein-based sealant for surgical applications, Science translational medicine 2017, 9(410) eaai7466). Porcine skin was cut into small rectangular pieces (1×2 cm), and the excess fat was removed. Tissues were moisturized with PBS before testing. The tissues were then fixed onto two pre-cut microscope glass slides (20 mm×50 mm) by Krazy glue. 10 mm space was kept between the slides using the porcine skin. The tissue was then separated in the middle with a straight edge razor to simulate the wound. 50 μL of prepolymer solution was injected onto the wound area and crosslinked by visible light. Maximum adhesive strength of each sample was obtained at the point of tearing at strain rate of 1 mm/min using a mechanical tester (n≥5).

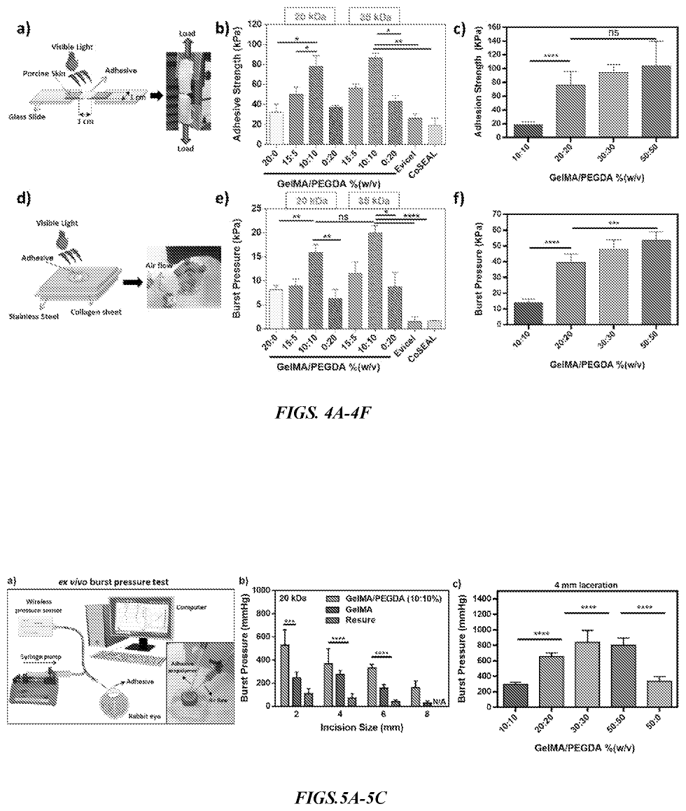

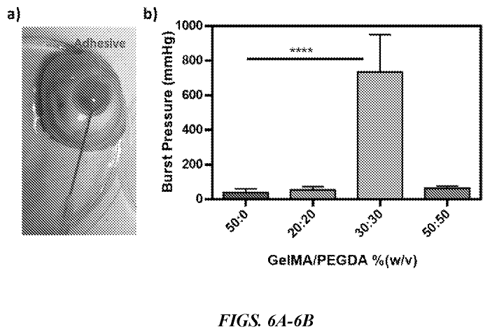

Ex vivo burst pressure test: Standard ex vivo tests were also performed to measure the burst pressures of rabbit corneas with full-thickness incisions after sealing with engineered bioadhesive and ReSure® as control (FIG. 5A). For the ex vivo tests, New Zealand rabbit eyes were explanted and full-thickness incisions with different sizes (2, 4, 6 and 8 mm) were created using surgical blade. The bioadhesive was then applied and photopolymerized to seal the incision. Afterwards, the sealed eye was connected to the burst pressure testing system, consisting of a pressure detection and recording unit and a syringe pump, that applied air with continuously increasing pressure towards the samples until bursting (FIG. 5A). The burst pressure was reported as the highest recorded pressure.

Ex vivo burst pressure test with liquid: A similar ex vivo burst pressure test was performed using 0.9% (w/v) saline solution as fluid. The burst pressures of rabbit corneas with full-thickness incisions (4 mm) after sealing with engineered bioadhesives was measured (FIG. 5A). The bioadhesive was applied and photopolymerized as described previously. Afterwards, the sealed eye was connected to the burst pressure testing system, consisting of a pressure detection and recording unit and a syringe pump, that applied saline solution with continuously increasing pressure towards the samples until bursting (FIG. 5A). The burst pressure was reported as the highest recorded pressure.

Slit Lamp Microscopy: Slit lamp microscopy was performed on explanted rabbit eyes using a Topcon system. Slit lamp photographs were also taken at the time of examination. With a 16× magnification, using slit and broad beams, transparency of the bioadhesive/defect area and surrounding cornea was evaluated using the Fantes grading scale (F. E. Fantes et al., Wound healing after excimer laser keratomileusis (photorefractive keratectomy) in monkeys, Archives of ophthalmology 108(5) (1990) 665-75), which is based on visibility of iris details.

Anterior Segment Optical Coherence Tomography: AS-OCT was performed on the rabbit eyes after application of bioadhesive to the laceration site. AS-OCT is a non-contact imaging modality that provides high-resolution cross-sectional images. A spectral-domain AS-OCT (Spectralis, Heidelberg Engineering, Germany), with an axial resolution of 3.9-7 μm, was used. Line scans (8 mm long) was performed at 0, 45, 90, and 135 degrees in the central cornea.

Statistical analysis: At least 3 samples were tested for all experiments, and all data were expressed as mean±standard deviation (*p<0.05, **p<0.01, ***p<0.001 and ****p<0.0001). T-test, one-way, or two-way ANOVA followed by Tukey's test or Bonferroni test were performed where appropriate to measure statistical significance (GraphPad Prism 6.0, GraphPad Software).

### Results and Discussion

Physical properties of the Engineered hybrid adhesive: Mechanical properties of GelMA/PEGDA adhesive hydrogels were characterized using tensile test. Tensile tests revealed that the elastic modulus (FIG. 1B) and extensibility (FIG. 1C) of the adhesive hydrogels could be modulated by varying the GelMA/PEGDA ratio and PEGDA molecular weight at a constant total polymer concentration. The elastic modulus of the composite adhesives was decreased significantly by changing the ratio of GelMA/PEGDA from 20/0 to 0/20). Although the elastic moduli of the engineered adhesives were lower than pure GelMA, the extensibility of the composite gels was significantly higher than GelMA (4.95-fold), when the concentration of GelMA/PEGDA was 10/10% (w/v) for both 20 kDa and 35 kDa PEGDA molecular weights. In addition, the extensibility of the composite hydrogels at this concentration was not significantly different from pure PEGDA samples (FIG. 1C).

According to FIG. 1D, the ultimate tensile strength of the composite adhesives was not significantly different compared to GelMA, when the concentration of GelMA/PEGDA was 10:10% (w/v). Overall, the mechanical properties of the adhesive gel show that the addition of PEGDA does not affect the ultimate tensile strength, while it remarkably increases the extensibility of the gels. This especially helps the flexibility and also cohesion of the material, since the extensibility and brittleness have an inverse relationship.

In vitro and ex vivo adhesion properties of the engineered adhesive hydrogels: To characterize the ability of GelMA/PEGDA hydrogels to seal wound boundaries upon tensile stress, in vitro wound closure tests were performed on native tissue, i.e. porcine skin, using ASTM F2458-05 standard (FIG. 4A) (Annabi, N. et al. Engineering a sprayable and elastic hydrogel adhesive with antimicrobial properties for wound healing, Biomaterials 2017, 139, 229-243). The adhesion strength for hydrogels at 20% (w/v) final polymer concentration was ranged between 37.2±5.3 kPa and 78.1±7.84 kPa by changing GelMA and PEGDA ratios for 20 kDa PEGDA (FIG. 4A). In addition, the adhesion strength of GelMA/PEGDA hydrogels (10:10% (w/v)) was 2.4-fold higher than pure GelMA. Similar behavior was observed for GelMA/PEGDA adhesives synthesized with 35 kDa PEGDA. Moreover, the adhesion strength for the hydrogel at 10:10% (w/v) GelMA/PEGDA ratio was 2.7-fold higher than GelMA hydrogel. This behavior can be due to higher cohesion strength of GelMA/PEGDA hydrogels compared to pure GelMA.

Next, to characterize the ability of GelMA/PEGDA adhesive to seal full thickness lacerations in the cornea, in vitro burst pressure tests were performed according to ASTM F2392-04 standard on a collagen substrate. The burst pressure resistance obtained for hydrogels at 20% (w/v) total polymer concentration and different GelMA/PEGDA concentrations ranged from 3.7±1.6 kPa to 15.9±2.1 kPa, for 20 kDa PEGDA (FIG. 4B). In addition, for both 20 kDa and 35 kDa PEGDA molecular weights, the GelMA/PEGDA hydrogels at 10:10% (w/v) showed remarkably higher adhesion strength compared to pure GelMA (2.0 and 2.5-fold respectively).

Overall, the adhesion properties of the engineered GelMA/PEGDA adhesives showed promising for closure of wounds on native porcine skin as well as sealing the small lacerations in the collagen sheets. The ability of the composite adhesives in sealing full thickness lacerations with different sizes in explanted rabbit eyes is next evaluated.

To allow for sutureless repair of corneal lacerations, a biocompatible and strong sealant is required which can stay on the cornea long enough for complete wound healing. Although the sealant ReSure® has been approved for sealing small corneal incisions after cataract surgery, it falls off quickly and is not designed for sealing traumatic corneal lacerations. In the ex vivo experiments (FIG. 5B), it was found that ReSure® could not seal full-thickness corneal incisions with diameters larger than 6 mm. In addition, both adhesive formulations, GelMA and GelMA/PEGDA, had much higher burst pressures compared with ReSure® for different sizes of full-thickness corneal incisions (FIG. 5B). For example, the burst pressure of the engineered GelMA was higher than 30.1±4.3 kPa, almost 10 times the pressure of a healthy eye, and significantly higher than the burst pressure of the commercial control, ReSure® (15.4±6.3 kPa) (FIG. 5B). Overall, the composite adhesive showed high capability to seal full-thickness corneal lacerations and it is expected to seal the lacerations for long enough to allow for complete healing of lacerations of different sizes.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A composition comprising acryloyl-substituted gelatin, acryloyl-substituted polyethylene glycol (PEG), and a visible light activated photoinitiator.

2. The composition of claim 1, wherein the composition further comprises a pharmaceutically acceptable carrier or excipient.

3. The composition of claim 1, wherein the composition comprises acryloyl-substituted gelatin in an amount from about 1% to about 40%, wherein the percent is a weight/volume percent, mass/volume percent, weight/weight percent, or mass/mass percent.

4. The composition of claim 1, wherein composition comprises acryloyl-substituted polyethylene glycol in an amount from about 1% to about 40%, wherein the percent is a weight/volume percent, mass/volume percent, weight/weight percent, or mass/mass percent.

5. The composition of claim 1, wherein the acryloyl-substituted gelatin and the acryloyl-substituted polyethylene glycol are present in a ratio from about 30:1 to about 1:30, wherein the ratio is weight to weight, mass to mass, or weight/volume percent to weight/volume percent.

6. The composition of claim 1, wherein the acryloyl-substituted gelatin is a methacryloyl-substituted gelatin.

7. The composition of claim 1, wherein the acryloyl-substituted gelatin has a degree of acryloyl substitution between about 50% and about 90%.

8. The composition of claim 1, wherein the acryloyl-substituted polyethylene glycol is diacrylated polyethylene glycol (PEGDA).

9. The composition of claim 1, wherein the composition comprises at least two different photoinitiators.

10. The composition of claim 1, wherein composition further comprises a therapeutic agent or a cell.

11. The composition of claim 10, wherein the cell is a corneal cell.

12. The composition of claim 11, wherein the composition is formulated for topical use.

13. A composition comprising acryloyl-substituted gelatin cross-linked with acryloyl-substituted polyethylene glycol.

14. The composition of claim 13, wherein the composition is in a form of a hydrogel.

15. The composition of claim 13, wherein the composition further comprises a pharmaceutically acceptable carrier or excipient.

16. The composition of claim 13, wherein the composition comprises acryloyl-substituted gelatin in an amount from about 1% to about 40%, wherein the percent is a weight/volume percent, mass/volume percent, weight/weight percent, or mass/mass percent.

17. The composition of claim 13, wherein composition comprises acryloyl-substituted polyethylene glycol in an amount from about 1% to about 40%, wherein the percent is a weight/volume percent, mass/volume percent, weight/weight percent, or mass/mass percent.

18. The composition of claim 13, wherein the acryloyl-substituted gelatin, acryloyl-substituted polyethylene glycol are present in a ratio from about 30:1 to about 1:30, wherein ratio is weight to weight, mass to mass, or weight/volume percent to weight/volume percent.

19. The composition of claim 13, wherein the acryloyl-substituted gelatin is methacryloyl-substituted gelatin.

20. The composition of claim 13, wherein acryloyl-substituted gelatin has a degree of acryloyl substitution between about 50% and about 90%.

21. The composition of claim 13, wherein the acryloyl-substituted polyethylene glycol is diacrylated polyethylene glycol.

22. The composition of claim 13, wherein composition further comprises a therapeutic agent or a cell.

23. The composition of claim 13, wherein the composition is formulated for topical use.

24. A method for treating a soft tissue injury or wound, comprising:
a. applying acryloyl-substituted gelatin, acryloyl-substituted polyethylene glycol (PEG), and a visible light-activated photoinitiator to the injury or wound; and
b. applying visible light to activate the photoinitiator, thereby cross-linking the acryloyl-substituted gelatin and the acryloyl-substituted PEG.

25.-38. (canceled)

39. A method for treating a soft tissue injury or wound, comprising applying a composition comprising acryloyl-substituted gelatin cross-linked with acryloyl-substituted polyethylene glycol.
